(19) United States
(12) Patent Application Publication
(10) Pub. No.: US 2004/0220220 A1
(43) Pub. Date: Nov. 4, 2004

(54) Method for treating inflammatory bowel disease

(76) Inventor: B. Lauren Charous, Fox Point, WI (US)

Correspondence Address: Scully, Scott, Murphy & Presser, 400 Garden City Plaza, Garden City, NY, 11530, US

(21) Appl. No.: 10/805,754

(22) Filed: Mar. 22, 2004

Related U.S. Application Data
(63) Continuation-in-part of application No. 10/779,522, filed on Feb. 13, 2004.
Application No. 10/779,522 is a continuation-in-part of application No. 10/293,769, filed on Nov. 12, 2002.
(60) Provisional application No. 60/345,877, filed on Nov. 9, 2001.

(51) Int. Cl. A61K 31/47

(52) U.S. Cl. 514/312; 514/313

(57) ABSTRACT

The present invention is directed to a method for treating inflammatory bowel disease in a patient comprising administering to said patient a pharmaceutical composition comprising a pharmaceutically effective amount of an anti-malarial compound in association with a pharmaceutically acceptable carrier and/or excipient that delays and targets the release of the anti-malarial compound in the gastrointestinal tract of the patient.

It is also directed to the pharmaceutical composition comprising a pharmaceutically effective amount of the anti-malarial compound in association with a pharmaceutically acceptable carrier or excipient that delays and target the release of the anti-malarial compound in the gastrointestinal tract.

### RELATED APPLICATION

The present application is a continuation in part of copending application U.S. Ser. No. 10/779,552 filed on Feb. 13, 2004, which is a continuation in part of copending application U.S. Ser. No. 10/293,769 filed Nov. 12, 2002 which is claiming priority of provisional application U.S. S. No. 60/345,877 filed on Nov. 9, 2001.

### FIELD OF THE INVENTION

The present invention relates to a method for treating inflammatory bowel disease with a novel oral preparation of anti-malarial agents.

### BACKGROUND OF INVENTION

Inflammatory bowel is an idiopathic illness characterized by a constellation of historical and physical findings as well as pathological lesions of the intestinal mucosa in which the sustained activation of mucosal immune responses play a major role. The major forms of inflammatory bowel disease include: Crohn's disease (CD) and Ulcerative colitis (UC). Another form of IBD is eosinophilic gastroenteritis (EG) which is much more rare.

The prevalence of Crohn's disease is 20-100 and UC 40-100 per 100,000. Eosinophilic gastroenteritis is more rare. The cause of all of these illnesses remains unknown despite much research. Although genetic factors may predispose individuals, and allergic disease appears frequently in individuals with EG, the frequency of disease in first-degree relatives argues against a simple “recessive” inheritance patterns for any of these illnesses.

Crohn's disease is predominantly a small-bowel disease with 30% of individuals having ileo-cecal disease; 40% with disease restricted to the small bowel and 30% with involvement of the colon. Complications of CD include severe diarrhea, abdominal pain, weight loss, malabsorption, intra-abdominal abscesses, intestinal fistulae and obstruction, gall stones, kidney stones and an increased incidence of colon cancer. The inflammation in CD chiefly involves macrophages, and activated T cells, although eosinophilic infiltrations are noted in many patients. Granulomatous changes are also seen in a minority of patients. While the inflammatory pathways of this illness remain to be fully elucidated, it is clear that pro-inflammatory mediators such as TNF-α, IL-1, IL-6 and interferon-γ play a major role in the pathogenesis of this illness.

Ulcerative colitis affects the colon and rectal mucosa and superficial submucosa. The inflammatory process involves neutrophilic infiltration of the lamina propria and intestinal crypts with frequent micro-abscess formation. A mixed-cell inflammatory change is commonly seen, with involvement of lymphocytes and other leukocytes, including at times prominent eosinophilic involvement with more extensive inflammation. Manifestations of UC include bloody diarrhea, abdominal and rectal pain, fever, weight loss and malaise. Complications of UC include colonic perforation, toxic megacolon, arthritis, and a marked increase in the risk of colon cancer and sclerosing cholangitis.

EG may affect any portion of the gastrointestinal tract, but most commonly involves the esophagus, stomach and small bowel. The illness is characterized by blood eosinophilia and eosinophilic infiltration of the gastrointestinal mucosa and underlying tissue. Activated eosinophils are capable of releasing a variety of cellular toxins, including eosinophil cationic proteins, superoxides, and eosinophil derived neurotoxin and eosinophil major basic protein. Eosinophils may also play a role in antigen presentation and ICAM-bearing eosinophils may have increased adhesion capacity for antologous T cells (Hansel T T, “Induction and function of eosinophil intercellular adhesion molecule-1 and HLA-DR”, J Immunol 1992; 149: 2130-6). Symptoms of EG may include pain, nausea, vomiting, diarrhea, but also intestinal obstruction and perforation.

In addition to leukocyte-mediated inflammation, increasing evidence mounts for the active participation of intestinal epithelial cells in the inflammatory process by their central role in leukocyte recruitment and activation. Epithelial cells when stimulated by pro-inflammatory cytokines produce and secrete potent, functional chemokines, such as IP-10 and RANTES which recruit leukocytes responsible for the secondary and ongoing inflammatory response. Up-regulation of the expression of the cell adhesion molecule receptors, ICAM-1, -2 and -3, on epithelial cells is believed to be pivotal for the migration of leukocytes from the circulation toward the colonic epithelium. Furthermore, the production and secretion of pro-inflammatory cytokines such as TNF-alpha, IL-1β, IL-8 and IL-6 by these cells plays a critical role in activating these leukocytes and ultimately in the maintenance of an inflammatory response (in a variety of experimental systems) (Uguccioni, M, et al. “Increased expression of IP-10, IL-8, MCP-1 and MCP-3 in ulcerative colitis”, Am J Pathol, 1999:155:231-6; Vainer B et al. “Comparative studies of the colonic in situ expression of intercellular adhesion molecules (ICAM-1, -2, -3) beta2 integrins (LFA-1, Mac-1, and p150,95) and PECAM in Ulcerative Colitis and Crohn's Disease”, Am J Surg Pathol. 2000;24:1115-24; Dwinell, M B et al., “Regulated production of interferon-inducible T-cell chemoattractants by human intestinal epithelial cells”, Gastroenterology, 2001; 120: 291-4; Lahav M et al., “Lidocaine inhibits secretion of IL-8 and IL-1 beta and stimulates secretion of IL-1 receptor antagonist by epithelial cells”, Clin Exp Immunol, 2002;127:226-33; Kirkegaard T et al., “Tumour necrosis factor-alpha converting enzyme (TAC8) activity in human colonic epithelial cells”, Clin Exp Immunol., 2004;135:146-53.

Treatment of IBD commonly utilizes a variety of oral systemic anti-inflammatory agents designed to reduce the inflammatory response. First line therapy commonly employs one of the 5-aminosalicylates, such as sulfasalazine, olsalazine or mesalamine.

Alternate anti-inflammatory agents generally accepted as treatment of IBD, include corticosteroids, azathioprine, cyclosporine and tacrolimus. In addition, methotrexate, the TNF-α blocker infliximab (Remicade®), and corticosteroids have been given parenterally via injection or intravenous infusion. Despite their considerable toxicity, oral and parenteral corticosteroids are considered the only proven treatment for the treatment of EG. With the exception of the 5-aminosalicylates, all of these therapeutic approaches rely on the administration of these drugs systemically and derive their benefit from the general anti-inflammatory effects which result.

Only two clinical trials of the anti-malarial agent Hydroxychloroquine (HCQ) given at conventional oral doses of from 4-6 mg/kg/day (typically 400 mg/day for a an average-sized person) are reported (Goenka, MK et al. Am J Gastroenterol 1996;91:917-21; Mayer, L, “The role of the epithelial cell in immunoregulation: pathogenetic and therapeutic implications”, Mt. Sinai J Med, 1990; 57: 179-82); in neither was a consistent therapeutic benefit evident after 3 months therapy. For this reason, current therapeutic recommendations for IBD do not include use of HCQ or other anti-malarial agents (Podolsky, DK, “Inflammatory bowel disease”, NEJM, 2002; 347: 417-29; Scholmerich, J, “Immunosuppresive treatment for refractory ulcerative colitis—where do we stand and where are we going?”, Eur J Gastroenterol Hepatol 1997; 9: 842-9).

The reason for an apparent lack of rapid, consistent effects of standard oral dosing with HCQ and other anti-malarial agents is not immediately clear. HCQ is known to have anti-inflammatory effects on many of the cells and pro-inflammatory chemokines involved in IBD. Furthermore, in a variety of ex vivo and in vitro experiments, HCQ does exert a very immediate effect on leukocytes, usually in less than 1 hour of incubation.

Despite this, HCQ and other anti-malarial agents are universally considered slow acting drugs. In the treatment of rheumatic diseases, such as lupus erythematosus and rheumatoid arthritis, onset of action is characteristically 3-4 months. Charous presented convincing evidence (Charous, BL et al., J Allerg Clin Immunol, 1998; 102: 198-203) that therapeutic effect in asthma with oral HCQ begins only after 24-26 weeks of treatment.

This delay in onset appears due to the requirement for active drug concentration in target organs for the onset of therapeutic effect. Hence, one requirement for drug action is time. The second requirement for onset of drug effect is that the anti-malarial compounds, e.g., HCQ, achieve therapeutic concentration in the target organs. Inasmuch as HCQ has a notable selective distribution throughout body organs (McChesney, EW, “Animal toxicity and pharmacokinetics of hydroxchloroquine sulfate”, Amer J Med, 1983; July: 11-18), administration by HCQ per ora does not imply that the sufficient drug concentrations will reach the inflamed interstinal mucosa. The fact that HCQ actively concentrates in organs other than bowel wall serves as a likely explanation for its lack of consistent and proven efficacy. It is noted that when given in conventional oral caplets, HCQ is not available directly to cells lining the esophagus due to its insoluble physical state following oral ingestion. Rather, it is liberated from the caplet in the stomach and virtually completely absorbed there and in the proximal bowel. For this reason, it cannot exert any direct effect from the gastrointestinal lumen on an inflammatory disease involving the esophageal, jejunal, ileal, cecal, colonic or rectal mucosa. In contrast, local delivery of the anti-malarial compounds, as defined herein, e.g., HCQ, can ensure that inflamed intestinal linings in the esophagus or in the distal small bowel, colon and proto-sigmoid are exposed to higher local drug concentrations than those achievable by any non-toxic conventional oral dosing regimen and which are sufficient to induce rapid onset of drug action.

### SUMMARY OF THE INVENTION

It is therefore an object of the present invention to provide a novel method for the administration of an anti-malarial agent as a localized enteric agent for treatment of diseased areas of the gastrointestinal tract. Specifically, the present invention is directed to the treatment of IBD, especially, Crohn's disease including Crohn's disease of the gastrointestinal tract from the mouth to the anus, and gastroduodenal Crohn's disease including but not limited to, ileocolitis, jejunoileitis, as well as Crohn's colitis, Crohn's perianal disease (such as proctitis) and proctosigmoiditis, of Ulcerative Colitis, including colitis, proctosigmoiditis and proctitis, of indeterminate colitis, of infectious colitis, of esophagitis and of ileitis and of Allergic and non-allergic eosinophilic esophagitis and gastroenteritis including involvement of the colon, said method comprising administrating a controlled, targeted release pharmaceutical composition comprising an anti-inflammatory effective amount of an anti-malarial compound to specific areas in the gastrointestinal tract (including small intestines, colon, rectum, and the like) involved in the inflammatory process. The anti-malarial compound may be associated with an excipient and/or carrier which controls and targets the release of the anti-malarial compounds at a targeted site of the gastrointestinal tract, e.g., esophagus, small intestine, large intestine, rectum, colon, small bowel and the like or a portion thereof. This release is controlled so that the anti-malarial compound is not released until it reaches a particular organ or portion thereof. Once the anti-malarial compound reaches the targeted site, the release thereof may be immediate, pulsed or it may be a sustained release, i.e., released over a prolonged period of time. Thus, the pharmaceutical composition may also comprise a sustained release carrier, such as a sustained release polymer known in the pharmaceutical arts. Alternatively, for treatment of esophageal diseases, an effective amount of the anti-malarial compound is associated with carriers known in the art that release drugs in the esophagus. For example, the anti-malarial compounds, disclosed herein, e.g., HCQ, can be associated with a carrier, such as syrup or other carrier, such as taste-masking system, known in the art which allows a therapeutic drug to be immediately released in the esophagus after swallowing. Alternatively, the anti-malarial compound can be associated with other carriers, which causes the drug to dissolve quickly in the mouth without chewing or the need for water. Such technologies referred to herein are exemplified in U.S. Pat. Nos. 6,641,838, 6,509,036, 6,391,335, 6,350,470 and U.S. Patent application 2003/0232080, the contents of all of which are incorporated by reference. Further, if the anti-malarial drug is associated with effervescent drug delivery system described in the aforementioned patents, it can be taste-masked using conventional techniques or as described in U.S. Patent Application 2003/0096791, the contents of which is incorporated by reference. Thus, for treatment of esophageal diseases, release of the is drug is controlled so that it occurs early while in transit from the mouth to the stomach so as to permit direct exposure of the esophageal lining to therapeutic drug concentrations. In particular, achievable drug concentrations in the intestinal lumen by use of targeted release can be shown to be of a magnitude previously shown to block eosinophil, neutrophil, macrophage and epithelial cell inflammatory responses.

The present inventor has found that the administration to the patient of the pharmaceutical composition comprising an anti-malarial compound with an excipient and/or carrier which controls and targets the release of the anti-malarial compound at the targeted site of a gastrointestinal tract results in unexpectedly rapid pharmacologic actions including suppression of inflammatory responses arising from eosinophilic infiltration of intestinal mucosa and bowel wall and of epithelial cell pro-inflammatory responses, all of which occur at surprisingly low drug doses.

Other aspects of the present invention are exemplified below.

Use of the compounds of the present invention for preventing and/or treating inflammatory bowel disease that manifests an increase in eosinophils.

Pharmaceutical compositions for preventing and/or treating inflammatory bowel disease that manifests an increase in eosinophils, which composition comprises the anti-malarial of the present invention and a pharmaceutically acceptable carrier.

This method has the advantage providing virtually immediate therapeutic drug concentrations to areas of inflammation in the intestines which will reduce the onset of action from months to days and decrease dosage requirements to 25% of conventional oral dosing.

It is another object of the present invention to demonstrate that this novel method for the administration of an anti-malarial agent provides rapid and surprising unsuspected anti-inflammatory effects on intestinal epithelial cells and on eosinophils.

It is another object of the present invention to provide a pharmaceutical composition comprising an anti-malarial compound in effective amounts in association with a sustained release carrier which releases the anti-malarial compound in the colon or small bowel.

### BRIEF DESCRIPTION OF THE DRAWING

FIG. 1 graphically depicts the effect of HCQ on eosinophil total superoxide production. In FIG. 1, PMA refers to phorbal myristic acetate, IL-5 refers to interleukin 5, and PAF refers to platelet activating factor, Nil refers to the control, i.e., no HCQ and SE refers to the standard error of the mean. The data are presented as mean ISE; n=3. The * indicates p<0.05; **indicates p<0.01.

FIG. 2 graphically depicts the effect of HCQ on IL-5 stimulated eosinophil degranulation as measured by release of eosinophil-derived neurotoxin. In this figure, EDN refers to eosinophil-derived neurotoxin.

FIG. 3 graphically depicts the effect of HCQ on IL-5 stimulated eosinophil survival. In the Figure, Dex refers to dexamethasone, and nil refers to IL-5 alone. The data are presented as average of duplicates from one or two experiments for each inhibitor/stimulus. Actual percentage survival at four days are given above each bar.

FIG. 4 graphically depicts the effects of varying concentrations of HCQ on IL-5 stimulated eosinophil survival. Apoptosis was examined by flow cytometry using annexin V-FITC and propidium iodide.

FIG. 5 graphically depicts the effect of 50 μM HCQ on the elaboration of IP-10 and RANTES in primary human epithelial cells with exposure to human rhinovirus HRV-16.

FIG. 6 graphically depicts the effects of HCQ on the release of RANTES in cultured human tracheal epithelial cells. In the figure, RV16-p3 refers to the rhinovirus strain. Differentiated cultures of human tracheal epithelial cells grown at an air liquid interface were pre-incubated for 12 hours with 50 μM HCQ prior to 12 hour infection with RV serotype 16. Twelve hours after removal of virus, levels of mRNA for RV were quantified using multiplex two-step RT-PCR.

FIG. 7 graphically depicts the effects of HCQ on the upregulation of ICAM-1 in cultured human tracheal epithelial cells. In the figure, RV16-p3 refers to the rhinovirus strain. ICAM refers to intracellular adhesion molecule 1. Differentiated cultures of human tracheal epithelial cells grown at an air liquid interface were pre-incubated for 12 hours with 50 μM HCQ prior to 12 hour infection with RV serotype 16. Twelve hours after removal of virus, levels of mRNA for RV were quantified using multiplex two-step RT-PCR.

FIG. 8 graphically depicts the effects of HCQ on the release of IL-1beta and TNF-alpha in cultured human tracheal epithelial cells. In the figure, RV16-p3 refers to the rhinovirus strain. IL-1beta refers to Interleukin-1 beta. TNF-alpha refers to Tumor Necrosis Factor-alpha. Differentiated cultures of human tracheal epithelial cells grown at an air liquid interface were pre-incubated for 12 hours with 50 μM HCQ prior to 12 hour infection with RV serotype 16. Twelve hours after removal of virus, levels of mRNA for RV were quantified using multiplex two-step RT-PCR.

FIG. 9 graphically depicts the effects of HCQ on the release of IL-6 in cultured human tracheal epithelial cells. In the figure, RV16-p3 refers to the rhinovirus strain. IL-6 refers to Interleukin-6. Differentiated cultures of human tracheal epithelial cells grown at an air liquid interface were pre-incubated for 12 hours with 50 μM HCQ prior to 12 hour infection with RV serotype 16. Twelve hours after removal of virus, levels of mRNA for RV were quantified using multiplex two-step RT-PCR.

FIG. 10 graphically depicts the effects of HCQ on the release of IL-8 in cultured human tracheal epithelial cells. In the figure, RV16-p3 refers to the rhinovirus strain. IL-8 refers to Interleukin-8. Differentiated cultures of human tracheal epithelial cells grown at an air liquid interface were pre-incubated for 12 hours with 50 μM HCQ prior to 12 hour infection with RV serotype 16. Twelve hours after removal of virus, levels of mRNA for RV were quantified using multiplex two-step RT-PCR.

FIG. 11 graphically depicts the mean whole blood concentration of HCQ following single day intravenous doses to male and female rats.

FIG. 12 graphically depicts the mean whole blood concentration of HCQ following single day intravenous doses to male and female dogs.

FIG. 13 depicts graphically the effect of varying concentrations of HCQ preincubation on the elaboration of IP-10 and RANTES in BEAS-2B epithelial cells exposed to HRV-16.

### DETAILED DESCRIPTION OF THE INVENTION

As used herein, the term “inflammatory bowel disease (“IBD”) includes inflammatory bowel disease of whatever-type, etiology or introgenesis. It includes, without limitation, ulcerative colitis, collagenous colitis, colitispolyposa, transmural colitis, Crohn's disease, indeterminate colitis, infectibous colitis, non-allergic eosinophilic esophogitis and eosinophilic gastroenteritis and the like.

As used herein, eosinophilic related disorder refers to a condition or disease in which there is the formation and accumulation of an abnormally large number of eosinophils in the tissues. The name of the disorder derives from easin, a rare-colored stain or dye comprising a bromine derivative of fluorescence which readily stains eosinophilic leukocytes in the blood of patents who are thus readily identified.

As used herein, Crohn's disease is characterized by eosinophilic tissue infiltration. It includes without limitation esophagitis, ileocolitis, jejunoileitis, colitis, perianal disease, proctosigmoiditis, and gastroduodenal Crohn's disease.

Ulcerative colitis is also characterized by eosinophilic tissue infiltration. It includes without limitation ileitis, proctosigmoiditis, and proctitis.

As used herein, “treating” refers to the administration of an anti-malarial compound at any time during or after the onset of at least one symptom of an inflammatory bowel disease in the patient to ameliorate or prevent from worsening at least one symptom of an inflammatory bowel disease. It, thus, has a therapeutic effect.

As used herein, the term prophylactic or synonym thereto refers to the prevention at any time prior to the onset of at least one symptom of inflammatory bowel disease in the patient, i.e., prophylactic therapy.

The present invention is directed to the use of an anti-malarial compound as defined herein, in association with pharmaceutically acceptable excipient which controls the release thereof at a targeted site of the gastrointestinal system for the treatment of inflammatory bowel disease in a patient suffering from same. As shown, hereinbelow, the anti-malarial compound is effective for inducing programmed cell death in eosinophils and thus, it is useful in treating an esoinophil mediated disease in which an excess of eosinophils appears in the tissue or blood, especially in relation to gastrointestinal disease, such as IBD as defined herein.

In addition, the present invention is directed to the use of an anti-malarial compound, as defined herein, in association with a pharmaceutically acceptable excipient which controls the release thereof at a targeted site of the gastrointestinal system for the prophylaxis of gastrointestinal diseases such as IBD. For example, the anti-malarial compound is administered to patients who may not manifest any symptoms of IBD, but who may be prone to being affected with IBD. For instance, the patient's family history may make him or her more prone to being afflicted with IBD. These functions or markers which make a person prone to being affected with IBD are known to one of ordinary skill in the art.

The prophylactically effective amount of treating IBD as well as the therapeutically effective amount can be determined by one of ordinary skill in the art. Preferably, the prophylactically effective amount is the same amount as the therapeutic effective amount, as defined hereinbelow.

The present inventor has discovered that an anti-malarial agent administered in a local or targeted fashion in a sustained release formulation, directly to the diseased organ or area of inflammation of a patient, is much more effective than when administered orally in a non-directed fashion, with the result that the drug has therapeutic utility at surprisingly low doses and with surprising rapidity in the targeted tissues or organs, while at the same time minimizing the risk of undesirable side-effects. Accordingly, the present inventor has discovered that an anti-malarial agent administered in a local or targeted fashion, directly to the diseased organ or area of inflammation of a mammal, e.g., patient, is much more effective and efficacious than when administered in a conventional oral dosage with the result that the agent reaches a therapeutic level with surprising rapidity, in the targeted tissue organ, while the undesirable side effects are minimized.

The present invention is illustrated by comparing the effects of targeted delivery as opposed to systemic delivery of a representative anti-malarial compound, HCQ, for treatment of EG. The following discussion is illustrative; it applies not only to HCQ, as shown below, but also the other anti-malarial compounds of the present invention.

### GENERAL PROCEDURE FOR EXAMPLES 1-4

The following protocols were generally used for Examples 1-4.

Human eosinophils were isolated from atopic and non-atopic donors using density centrifugation and CD16-magnetic bead negative selection. Eosinophil purity was always>93%.

Flow cytometry was performed on a Becton Dickenson FACScan. Data was analyzed using CellQuest software. For cell surface marker determination, specific antibodies directly conjugated with the fluorescent indicator, phycoerythrin (PE) were utilized.

Statistical analyses were performed using the Student's paired t-test; significance defined as p value<0.05 compared to “nil” or 0.0 microM HCQ.

### Example 1

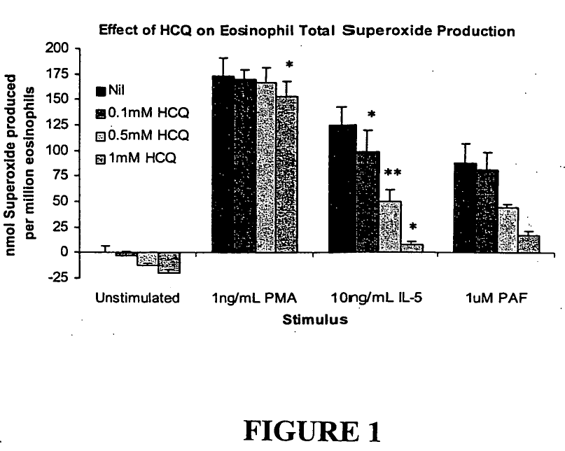

The general procedure described hereinabove was followed. Eosinophils were pretreated with HCQ for 1 hr at 37° C. and then stimulated as described in FIG. 1 in 96-well plates (50,000 cells/well). Extracellular superoxide was detected using cytochrome c and a 96-well plate reader.

As seen in FIG. 1, IL-5 or PAF induced eosinophil superoxide is inhibited by HCQ but only at concentrations of at least 0.5 mM, or about 200 mcg/ml. Similarly, eosinophil degranulation is inhibited by HCQ at concentrations in excess of about 100 microM.

### Example 2

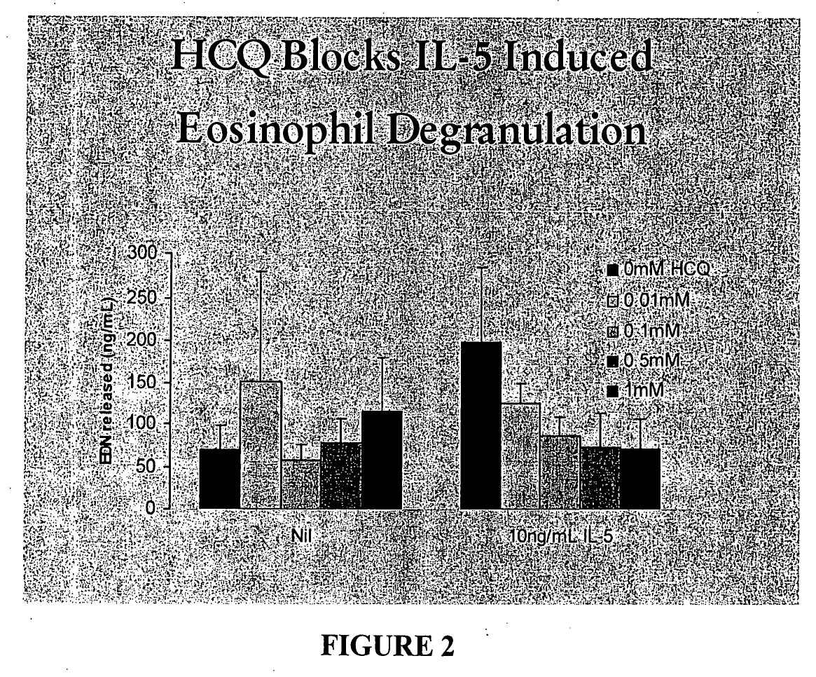

The general procedure described hereinabove and Example 1 was followed. Supernatants from superoxide assay samples were analyzed for EDN by Double-Antibody Radioimmunoassay (DARIA). The results are depicted in FIG. 2.

### Example 3

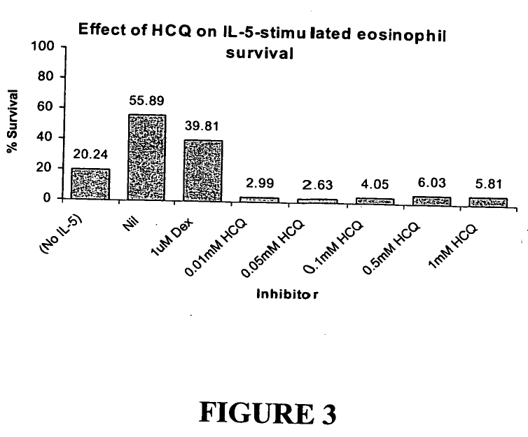

The general procedure described hereinabove was followed. Eosinophils were incubated with 100 pg/ml IL-5 with or without dexamethasone or HCQ. After four days, eosinophil viability was measured by flow cytometry. The results are depicted in FIG. 3.

Furthermore, as seen in FIG. 3, HCQ actively shortens eosinophil survival, to a much greater extent than a comparative dose of dexamethasone, a corticosteroid.

### Example 4

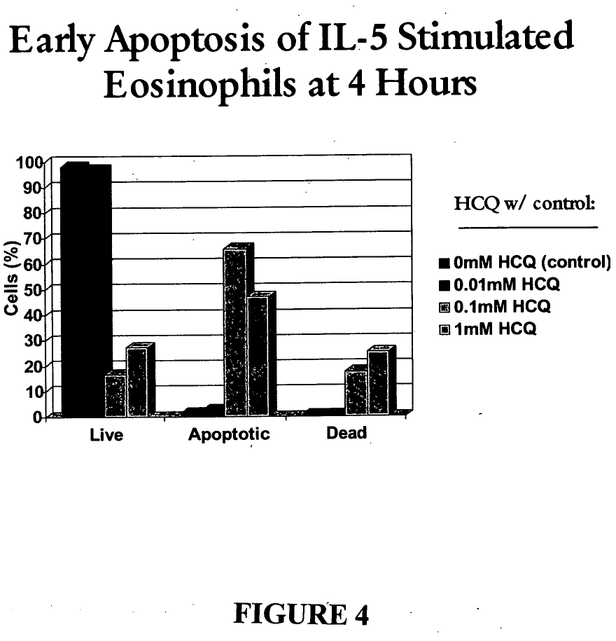

The general procedure described hereinabove was followed. Eosinophils were incubated with 100 pg/ml IL-5 with or without HCQ. At predetermined times, eosinophils were stained with Annexin-V-FITC and propidium iodide. Live cells were defined as double negative, apoptotic cells were defined as Annexin-V-FITC positive only, and dead cells were defined as Annexin-V-FITC and propidium iodide double positive. The results are depicted in FIG. 4.

As shown in FIG. 4., HCQ induces eosinophil apoptosis (programmed cell death) at concentrations between about 10 and about 100 microM. These effects on eosinophils are nearly immediate and require only 1 hour pre-incubation.

Similar rapid effects of effects of targeted delivery as opposed to systemic delivery of a representative anti-malarial compound, HCQ, are seen in epithelial cells. Rhirovirus infection is a method to induce reproducible epithelial pro-inflammatory responses, including elaboration of chemokines and cytokines.

### Example 5

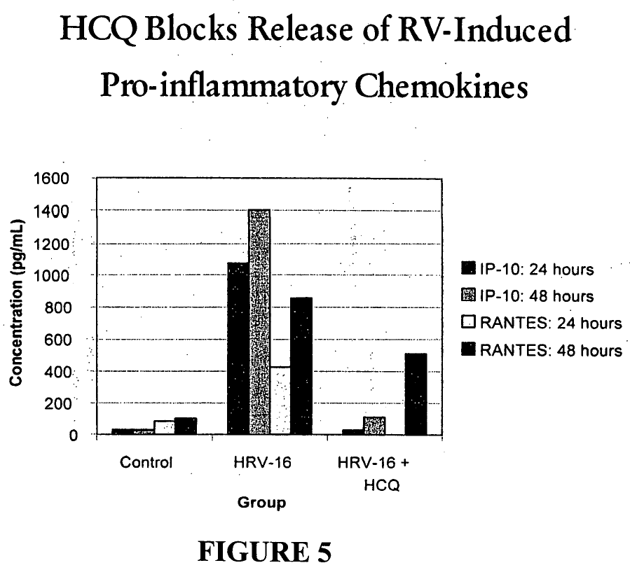

Primary cultures of harvested human tonsillar epithelial cells were preincubated for 24 hr with 50 microM HCQ. The cells were then exposed to human rhinovirus type 16 (HRV-16) which was left on the cells in the presence and absence of HCQ. Medium was recovered at two different time post infection (24 hr viral incubation and 48 hr viral incubation) and assayed for IP-10 and RANTES. The data is depicted in Table 1 below and in FIG. 5.

TABLE 1
Effect of HCQ on IP-10/RANTES Production in Primary
Human Epithelial Cells in pg/ml with exposure to HRV-16

| | IP-10: hrs of | | | RANTES: hrs of | |
|---|---|---|---|---|---|
| | preincubation | | | preincubation | |
| Experiment | 24 | 48 | Experiment | 24 | 48 |
| Control | 31 | 31 | Control | 86 | 104 |
| HRV-16 | 1075 | 1400 | HRV-16 | 425 | 854 |
| HRV-16 + 50 | 31 | 112 | HRV-16 + HCQ | ND | 509 |
| microM HCQ | | | | | |

### Example 6

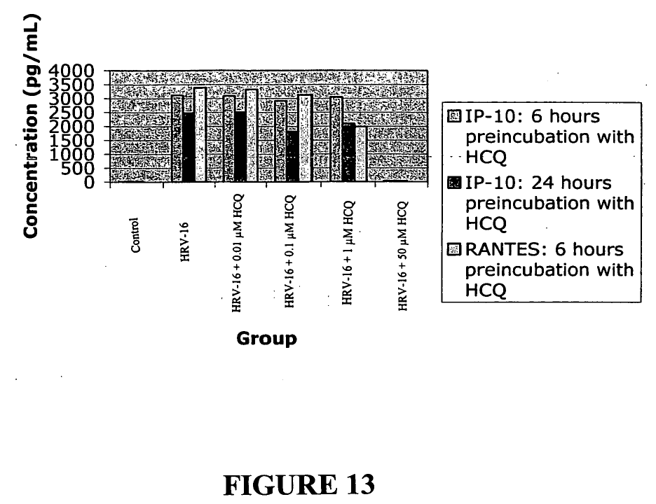

Cells of the BEAS-2B epithelial cell line were preincubated for either 6 hr or 24 hr with varying concentrations of HCQ. Cells were then exposed to to human rhinovirus type 16 (HRV-16) which was left on the cells in the presence and absence of HCQ at the concentrations indicated for 48 hr. The data is depicted in Table 2 and FIG. 13.

TABLE 2
Effect of varying concentrations of HCQ preincubation on
BEAS-2B epithelial cells in pg/ml exposed to HRV-16 and
assayed for IP-10 and RANTES

| | IP-10: 6 hrs | IP-10: 24 hrs | RANTES: 6 hrs |
|---|---|---|---|
| | preincubation | preincubation | preincubation |
| Control | 31 | 31 | 0 |
| HRV-16 | 3123 | 2478 | 3388 |
| HRV-16 + 0.01 mM | 3084 | 2506 | 3326 |
| HCQ | | | |
| HRV-16 + 0.1 mM | 2914 | 1814 | 3128 |
| HCQ | | | |
| HRV-16 + 1 mM | 3045 | 2098 | 1994 |
| HCQ | | | |
| HRV-16 + 50 mM | 31 | 31 | 0 |
| HCQ | | | |

As shown by the data, at effective concentrations, e.g., 50 microM to about 250 microM, epithelial cell production of pro-inflammatory cytokines such as IP-10 and RANTES are also inhibited by HCQ (See Tables 1, 2; FIGS. 5, 13).

### Examples 7-11

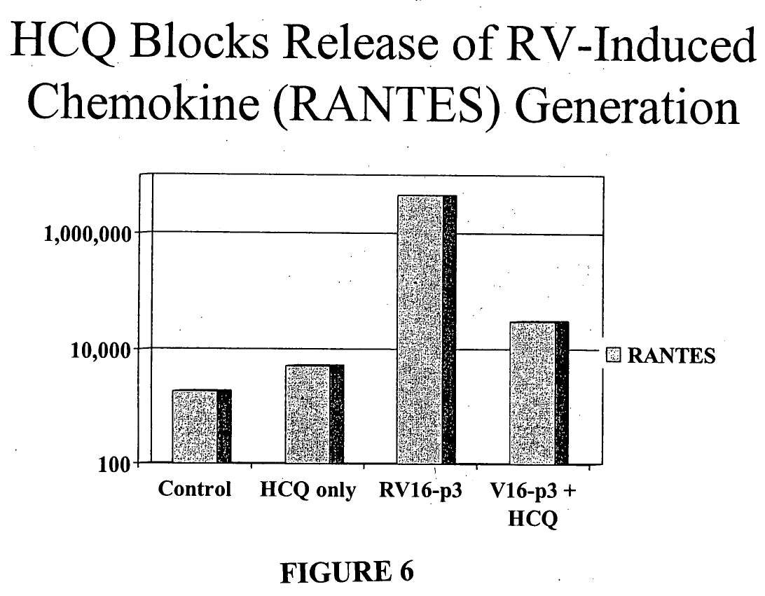

Differentiated cultures of human tracheal cells grown at an air liquid interface were pre-incubated for 12h with 50 microM HCQ prior to 12 h infection with human rhinovirus type 16 (HRV-16). Twelve hours after removal of virus, levels of mRNA for RANTES, ICAM-1, IL-1beta, TNF-alpha and IL-6 were quantified using multiplex two-step (TaqMan) RT-PCR. The results are depicted in FIGS. 6-10.

FIG. 6 graphically depicts the effects of HCQ on the release of RANTES (regulated on activation normal T-cell expressed and secreted) in cultured human tracheal epithelial cells. In the figure, RV16-p3 refers to the rhinovirus strain. Human tracheal epithelial cells were cultured for 14 days in serum-free growth media. On day 14, the cultures were divided into four groups. Two groups were pre-treated for 6 hours with HCQ (final concentration 50 microM) in media administered via the basolateral side. Then RV-16-p3 at a tissue culture infectious dose (TCID₅₀) of 10⁶/ml was added to one of groups receiving HCQ and one of thgroups not pretreated with HCQ. Phosphate buffered saline (PBS) was added to the other two groups, creating four conditions: media alone (control), HCQ alone, RV-16-p3 alone and RV-16-p3+HCQ. After six hours, the experimental suspensions were removed and the cells were washed three times with PBS. Cells were allowed to recover for 24h and then the mucosal surfaces of the culture inserts were flushed with 500 microL of PBS and the flushings were retained.

The filters containing the cell sheets were cut in half and placed in RLT buffer (Rneasy lysis buffer) or fixed in 10% buffered formalin prior to their preparation for fluorescence in situ hybridization (FISH) and gene expression analysis as described in detail elsewhere (Lopez-Souza N, et al., Resistance of differentiated human airway epithelium to infection by rhinovirus, Am J Physiol Lung Cell Mol Physiol, 2004;286:L373-81). Levels of mRNA for RANTES were determined in cell lysates using a two-step method of gene transcript profiling in which multiplex RT-PCR is combined with individual gene quantification via real-time PCR on generated cDNA product.

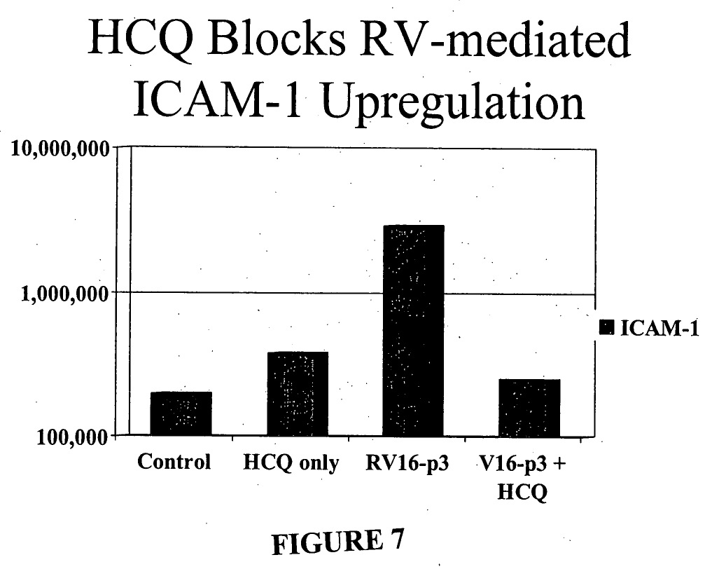

FIG. 7 graphically depicts the effects of HCQ on the upregulation of for intercellular adhision molecule 1 (ICAM-1) in cultured human tracheal epithelial cells. In the figure, RV16-p3 refers to the rhinovirus strain. ICAM refers to intracellular adhesion molecule 1. Human tracheal epithelial cells were cultured for 14 days in serum-free growth media. On day 14, the cultures were divided into four groups. Two groups were pre-treated for 6 hours with HCQ (final concentration 50 microM) in media administered via the basolateral side. Then RV-16-p3 at a tissue culture infectious dose (TCID₅₀) of 10⁶/ml was added to one of groups receiving HCQ and one of the groups not pretreated with HCQ. Phosphate buffered saline (PBS) was added to the other two groups, creating four conditions: media alone (control), HCQ alone, RV-16-p3 alone and RV-16-p3+HCQ. After six hours, the experimental suspensions were removed and the cells were washed three times with PBS. Cells were allowed to recover for 24h and then the mucosal surfaces of the culture inserts were flushed with 500 microL of PBS and the flushings were retained.

The filters containing the cell sheets were cut in half and placed in RLT buffer (Rneasy lysis buffer) or fixed in 10% buffered formalin prior to their preparation for fluorescence in situ hybridization (FISH) and gene expression analysis as described in detail elsewhere (Lopez-Souza N, et al., Resistance of differentiated human airway epithelium to infection by rhinovirus, Am J Physiol Lung Cell Mol Physiol, 2004;286:L373-81). Levels of mRNA for ICAM-1 were determined in cell lysates using a two-step method of gene transcript profiling in which multiplex RT-PCR is combined with individual gene quantification via real-time PCR on generated cDNA product.

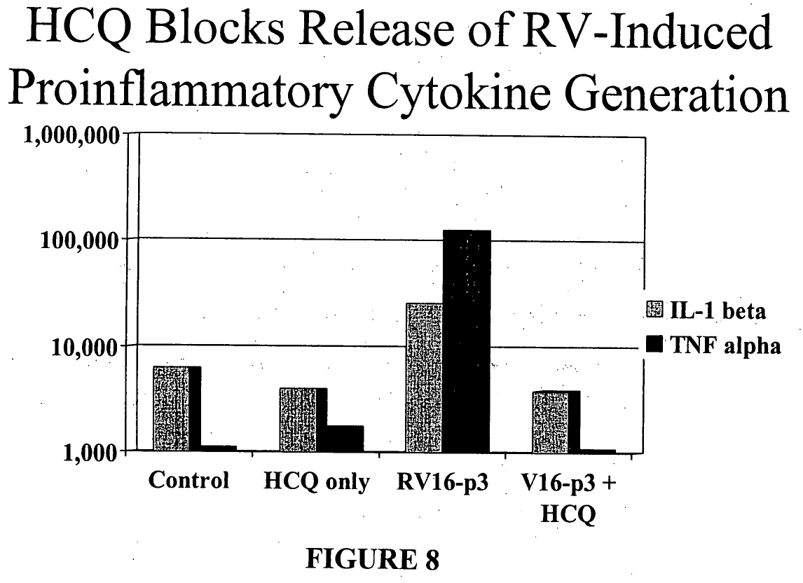

FIG. 8 graphically depicts the effects of HCQ on the release of IL-1beta and TNF-alpha in cultured human tracheal epithelial cells. In the figure, RV16-p3 refers to the rhinovirus strain. IL-1 beta refers to Interleukin-1 beta. TNF-alpha refers to Tumor Necrosis Factor-alpha. Human tracheal epithelial cells were cultured for 14 days in serum-free growth media. On day 14, the cultures were divided into four groups. Two groups were pre-treated for 6 hours with HCQ (final concentration 50 microM) in media administered via the basolateral side. Then RV-16-p3 at a tissue culture infectious dose (TCID₅₀) of 10⁶/ml was added to one of groups receiving HCQ and one of the groups not pretreated with HCQ. Phosphate buffered saline (PBS) was added to the other two groups, creating four conditions: media alone (control), HCQ alone, RV-16-p3 alone and RV-16-p3+HCQ. After six hours, the experimental suspensions were removed and the cells were washed three times with PBS. Cells were allowed to recover for 24h and then the mucosal surfaces of the culture inserts were flushed with 500 microL of PBS and the flushings were retained.

The filters containing the cell sheets were cut in half and placed in RLT buffer (Rneasy lysis buffer) or fixed in 10% buffered formalin prior to their preparation for fluorescence in situ hybridization (FISH) and gene expression analysis as described in detail elsewhere (Lopez-Souza N, et al., Resistance of differentiated human airway epithelium to infection by rhinovirus, Am J Physiol Lung Cell Mol Physiol, 2004;286:L373-81). Levels of mRNA for IL-1beta and TNF-alpha were determined in cell lysates using a two-step method of gene transcript profiling in which multiplex RT-PCR is combined with individual gene quantification via real-time PCR on generated cDNA product.

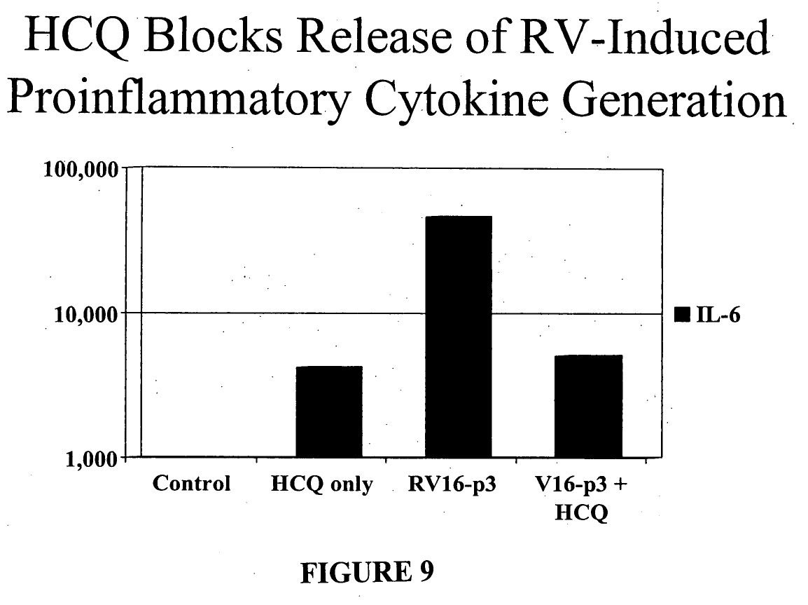

FIG. 9 graphically depicts the effects of HCQ on the release of IL-6 in cultured human tracheal epithelial cells. In the figure, RV16-p3 refers to the rhinovirus strain. IL-6 refers to Interleukin-6. Human tracheal epithelial cells were cultured for 14 days in serum-free growth media. On day 14, the cultures were divided into four groups. Two groups were pre-treated for 6 hours with HCQ (final concentration 50 microM) in media administered via the basolateral side. Then RV-16-p3 at a tissue culture infectious dose (TCID₅₀) of 10⁶/ml was added to one of groups receiving HCQ and one of the groups not pretreated with HCQ. Phosphate buffered saline (PBS) was added to the other two groups, creating four conditions: media alone (control), HCQ alone, RV-16-p3 alone and RV-16-p3+HCQ. After six hours, the experimental suspensions were removed and the cells were washed three times with PBS. Cells were allowed to recover for 24h and then the mucosal surfaces of the culture inserts were flushed with 500 microL of PBS and the flushings were retained.

The filters containing the cell sheets were cut in half and placed in RLT buffer (Rneasy lysis buffer) or fixed in 10% buffered formalin prior to their preparation for fluorescence in situ hybridization (FISH) and gene expression analysis as described in detail elsewhere (Lopez-Souza N, et al., Resistance of differentiated human airway epithelium to infection by rhinovirus, Am J Physiol Lung Cell Mol Physiol, 2004;286:L373-81). Levels of mRNA for IL-6 were determined in cell lysates using a two-step method of gene transcript profiling in which multiplex RT-PCR is combined with individual gene quantification via real-time PCR on generated cDNA product.

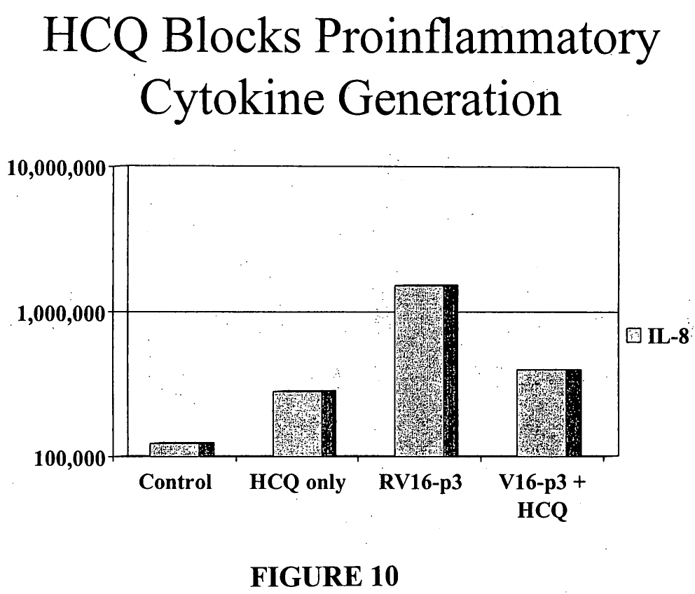

FIG. 10 graphically depicts the effects of HCQ on the release of IL-8 in cultured human tracheal epithelial cells. In the figure, RV16-p3 refers to the rhinovirus strain. IL-8 refers to Interleukin-8. Human tracheal epithelial cells were cultured for 14 days in serum-free growth media. On day 14, the cultures were divided into four groups. Two groups were pre-treated for 6 hours with HCQ (final concentration 50 microM) in media administered via the basolateral side. Then RV-16-p3 at a tissue culture infectious dose (TCID₅₀) of 10 ⁶/ml was added to one of groups receiving HCQ and one of the groups not pretreated with HCQ. Phosphate buffered saline (PBS) was added to the other two groups, creating four conditions: media alone (control), HCQ alone, RV-16-p3 alone and RV-16-p3+HCQ. After six hours, the experimental suspensions were removed and the cells were washed three times with PBS. Cells were allowed to recover for 24h and then the mucosal surfaces of the culture inserts were flushed with 500 microL of PBS and the flushings were retained.

The filters containing the cell sheets were cut in half and placed in RLT buffer (Rneasy lysis buffer) or fixed in 10% buffered formalin prior to their preparation for fluorescence in situ hybridization (FISH) and gene expression analysis as described in detail elsewhere (Lopez-Souza N, et al., Resistance of differentiated human airway epithelium to infection by rhinovirus, Am J Physiol Lung Cell Mol Physiol, 2004;286:L373-81). Levels of mRNA for IL-8 were determined in cell lysates using a two-step method of gene transcript profiling in which multiplex RT-PCR is combined with individual gene quantification via real-time PCR on generated cDNA product.

In these further studies, it is seen that effective concentrations of HCQ (e.g., 50 microM) rapidly block other pro-inflammatory responses by epithelial cells including up-regulation of ICAM-1 and release of TNF-alpha, IL-1beta, IL-6 and IL-8 (FIGS. 6, 7, 8, 9, 10).

On the other hand, oral or systemic administration of HCQ cannot provide adequate plasma levels of HCQ to achieve these effects is shown by the following experiments.

### Comparative Example 1

### Single-Dose Intravenous HCQ in Rats

Twelve rats per dose group (six M: six F) received hydroxychloroquine sulfate, USP as an intravenous bolus injected over 30 minutes.

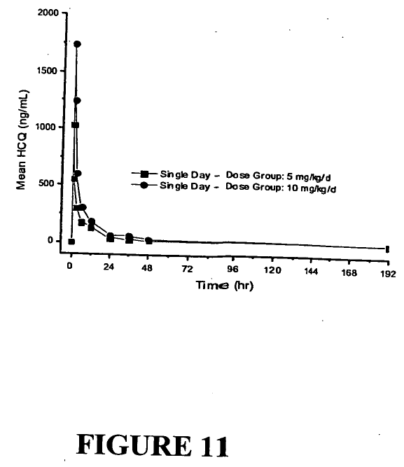

Following the single injection, blood samples were drawn from rat pairs at one, three, six, 12, 24, 36, 48, 192, 360, and 1,032 hours. The pharmacokinetic parameters are provided below in table 3, and the profiles of both doses are provided in FIG. 11.

TABLE 3
Summary of Pharmacokinetic Parameters of HCQ Following Single-Day
Intravenous Doses to Male and Female Rats

| | | | | | | | |
|---|---|---|---|---|---|---|---|
| | | | | | | | Apparent |
| Dose | AUC₀₋₂₃ | AUC_{0-∞} | V_{d} | CL | Cₘₐₓ | Tₘₐₓ | Terminal |
| (mg/kg/day) | (ng × hr/mL) | (ng × hr/mL) | (L/kg) | (L/kg-hr) | (ng/mL) | (hr) | t_{1/2}^{†}(hr) |

Single-Day Administration

| | | | | | | | |
|---|---|---|---|---|---|---|---|
| 5 | 4051.5 | 6563.0 | 11.1 | 1.2 | 1023.8 | 0.5 | 7.5 |
| 10 | 7224.5 | 10994.7 | 11.2 | 1.3 | 1735.0 | 0.5 | 7.4 |

^{†}2-compartment models were used to calculate the terminal half-life values.

### Comparative Example 2

### Single-Dose Intravenous HCQ in Dogs

Two Beagle dogs per dose group (one M: one F) received hydroxychloroquine sulfate, USP as an intravenous bolus infusion over 30 minutes.

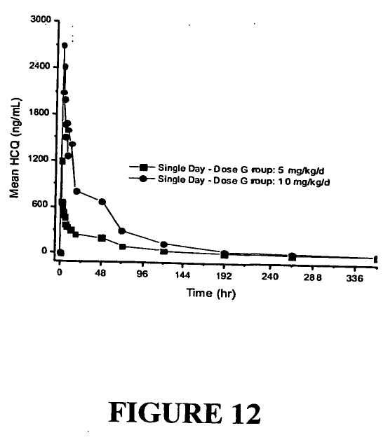

Following the single injection, blood samples were drawn at one, two, three, five, six, seven, eight, 12, 18, 24, 36, 120, 192, 264, 360, 528, 696, 864 and 1,032 hours. The pharmacokinetic parameters are provided below in Table 4, and the profiles of both doses are provided in FIG. 12.

TABLE 4
Summary of Pharmacokinetic Parameters of HCQ Following Single
Day Intravenous Doses to Male and Female Dogs

| | | | | | | | |
|---|---|---|---|---|---|---|---|
| | | | | | | | Apparent |
| Dose | AUC₀₋₂₃ | AUC_{0-∞} | V_{d} | CL | Cₘₐₓ | Tₘₐₓ | terminal |
| (mg/kg/day) | (ng · hr/mL) | (ng · hr/mL) | (L/kg) | (L/kg-hr) | (ng/mL) | (hr) | t_{1/2}^{†}(hr) |

Single-Day Administration

| | | | | | | | |
|---|---|---|---|---|---|---|---|
| 5 | 8157.6 | 24222.0 | 9.1 | 0.3 | 1195.0 | 0.5 | 20.8 |
| 10 | 30908.9 | 82058.0 | 6.1 | 0.1 | 2700.0 | 0.5 | 35.0 |

^{†}2-compartment models were used to calculate the terminal half-life values.

As shown by the results in FIGS. 11 and 12, even at doses nearly twice that used in humans, peak serum concentrations following intravenous administration of 10 mg/kg HCQ in rats was only 2 mcg/ml. See FIG. 11; in dogs, peak whole blood concentrations were less than 3 mcg/ml. See FIG. 12. These concentrations are approximately 1/100 of those required.

In contrast, targeted treatment of a section of the intestine with HCQ can easily reach therapeutic concentrations. For example, an 80 mg dose (20% of the conventional oral dose) delivered to the small bowel at a capacity generally estimated at 400 ml generously provides a lumenal drug concentration of over 200 microgram/ml (or >400 micromolar), considerably above that which has been shown to have the anti-inflammatory effects described above.

As shown in the literature, neutrophil and macrophage superoxide release as well as release by macrophages of potent chemokines such as TNF-alpha, IL-6, Interferon-gamma and T cell activation are also inhibited by HCQ at concentrations in this same range that were obtained by a targeted method of the present invention (See, NP Hurst Biochem Pharm 1986; 35: 3083-89; NP Hurst Annals Rheum Dis 1987; 46: 750-56, BEEM van den Borne J Rheumatol 1997; 24: 55-60; F Goldman Blood 2000; 95: 3460-66;Sperber K et al., “Selective regulation of cytokine secretion by hydroxychloroquine inhibition of interleukin 1 alpha (IL-1 alpha) and IL-6 in human monocytes and T cells”, J Rheumatol 1993; 20: 803-08).

The rapid effect of anti-malarials, especially those of the aminoquinoline class, on suppressing eosinophil and epithelial cell inflammatory responses has not been previously demonstrated (see FIGS. 1, 2, 5, 6, 7, 8, 9, 10). In particular the prompt induction of apoptosis, rather than necrosis, in eosinophils allows consideration of this category of drugs for treatment of inflammatory bowel disease since uncontrolled eosinophil death would heighten the likelihood of an adverse reaction to treatment (see FIGS. 3, 4). The therapeutic impact of these actions however rely on the targeted delivery of these agents so as to assure that therapeutic drug levels are present.

In summary, targeted delivery of the anti-malarial compounds, such as HCQ can provide a high and therapeutic lumenal drug concentration which cannot be matched by oral or parenteral drug administration.

These data clearly illustrate that the anti-malarial compounds of the present invention, including HCQ are useful in effective concentrations, as defined herein in treating eosinophil mediated diseases or conditions, i.e., a disease or condition in which results in an abnormal amount of esoinophils to appear in the blood, especially in relation to treating or preventing gastrointestinal diseases, such as IBD.

Eosinophilic tissue infiltration may accompany or result from a patient who is suffering from IBD. Thus, a patient is diagnosed with IBD, the administration of an anti-malarial compound, as described herein, especially with a carrier or excipient that targets the release of the compound in the gastrointestinal tract can be used to prevent or treat the onset of eosinophilic tissue infiltration as well as IBD. If a patient is diagnosed with IBD prior to being afflicted with eosinophilic tissue infiltration, a method of preventing eosinophilic tissue infiltration is by administering a prophylactic amount of the anti-malarial compound, as described herein, preferably with a carrier or excipient that target or controls the release thereof at the targeted site of the gastrointestinal tract. On the other hand, if the patient has eosinophilic tissue infiltration, especially resulting from the occurrence of IBD, then the eosinophilic tissue infiltration can be treated by administering to said patient a therapeutically effective amount of an anti-malarial compound as described herein, preferably with a carrier or excipient that targets and controls the release thereof at the targeted site in the gastrointestinal tract.

Using models which measure eosinophil function induced or perpetuated by IL-5, the actions of HCQ on eosinophil function was evaluated by various parameters including eosinophil survival, migration, and mediator release.

Accordingly, the present inventor has discovered that an anti-malarial agent administered in a local or targeted fashion, directly to the diseased organ or area of inflammation of a mammal, e.g., patient, is much more effective and efficacious than when administered orally with the result that the agent reaches a therapeutic level with surprising rapidity, in the targeted tissue or organ, while undesirable side effects are minimized. By anti-malarial, as used herein, it is meant that the drug has historically belonged to the class of drugs known as anti-malarials. Preferred anti-malarials include quinolines, especially 8 and 4 aminoquinolines, acridines, e.g., 9-amino acridines and quinoline methanols, e.g., 4-quinolinemethanols.

As used herein, the patient is a mammal. By mammal, it is meant a member of the class mammalia of higher vertebrate that have mammary glands and the females thereof have the ability to nourish their young with milk secreted by mammary glands. Examples of mammals includes cat, dog, horse, monkey, sheep, goat, cow, human and the like. The preferred mammal is human. The preferred patient is human.

Compounds suitable for the present invention are anti-malarial agents that have immunomodulatory and anti-inflammatory effects. Anti-malarial agents are well known in the art. Examples of anti-malarial agents can be found, for example, in GOODMAN AND GILMAN'S: THE PHARMACOLOGICAL BASIS OF THERAPEUTICS, chapters 45-47, pages 1029-65 (MacMillan Publishing Co. 1985), hereby incorporated by reference.

The preferred anti-malarial compounds are quinolines and more preferably aminoquinolines, especially 4- and 8-amino quinolines. An especially preferred class of antimalarials has a core quinoline structure (examples are mefloquine and quinine) which is usually substituted at one or more positions, typically at least at the 4- and/or 8-positions. One skilled in the art would understand that such agents could be administered in derivatized forms, such as pharmaceutically acceptable salts, or in a form that improves their pharmacodynamic profiles, such as esterification of acid or alcohol substituents with lower alkyls (e.g., C₁₋₆) or lower alkanoyloxy

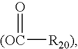

respectively, wherein R₂₀ is lower alkyl. Another class of antimalarials, exemplified by quinacrine, is based on an acridine ring structure, and may be substituted in the manner described above.

Especially preferred compounds for use in the present invention are aminoquinolines, including 4-amino and 8-aminoquinolines and their derivatives (collectively, “aminoquinoline derivatives”) and aminoacridines, especially 9-amino acridines. The preferred 4- and 8 aminoquinolines and 9-amino acridines are described by the following formula:

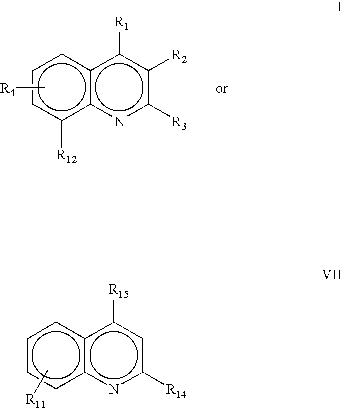

o pharmaceutically acceptable salts thereof, wherein

R₂ and R₃ are independently hydrogen, or lower alkyl or R₂ and R₃ taken together with the carbon atoms to which they are attached form an aryl ring, which ring may be unsubstituted or substituted with an electron withdrawing group or an electron donating group,

one of R₁ and R₁₂ is NHR₁₃ while the other is hydrogen;

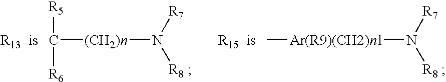

R₄, R₁₀, R₁₁ and R₁₄ are independently hydrogen or an electron donating group or electron withdrawing group;

R₅ and R₆, are independently hydrogen or lower alkyl which may be unsubstituted or substituted with an electron withdrawing or electron donating group;

R₇ and R₈ are independently hydrogen or lower alkyl, which may be unsubstituted or substituted with an electron withdrawing or electron donating group;

Ar is aryl having 6-18 ring carbon atoms;

R₉ is hydrogen or hydroxy or lower alkoxy or

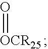

R₂₅ is lower alkyl or hydrogen; and

n and n₁ are independently 1-6.

As used herein, the terms “electron donating groups” and “electron withdrawing groups” refer to the ability of a substituent to donate or withdraw an electron relative to that of hydrogen if the hydrogen atom occupied the same position in the molecule. These terms are well understood by one skilled in the art and are discussed in Advanced Organic Chemistry, by J. March, John Wiley & Sons, New York, N.Y., pp. 16-18 (1985) and the discussion therein is incorporated herein by reference. Electron withdrawing groups include halo, including bromo, fluoro, chloro, iodo and the like; nitro; carboxy; carbalkoxy; lower alkenyl; lower alkynyl; formyl; carboamido; aryl; quaternary ammonium compounds, and the like. Electron donating groups include such groups as hydroxy; lower alkoxy; including methoxy; ethoxy and the like; lower alkyl, such as methyl; ethyl, and the like; amino; lower alkylamino; diloweralkylamino; aryloxy, such as phenoxy and the like; arylalkoxy, such as benzyl and the like; mercapto, alkylthio, and the like. One skilled in the art will appreciate that the aforesaid substituent may have electron donating or electron withdrawing properties under different chemical conditions. The term lower alkyl, when used alone or in conjunction with other groups, refers to an alkyl group containing one to six carbon atoms. It may be straight-chained or branched. Examples include methyl, ethyl, propyl, isopropyl, butyl, sec-butyl, isobutyl, tert-butyl, pentyl, neopentyl, hexyl and the like.

Lower alkoxy refers to an alkyl group which is attached to the main chain by an oxygen bridging atom. Examples include methoxy, ethoxy, and the like.

Lower alkenyl is an alkenyl group containing from 2 to 6 carbon atoms and at least one double bond. These groups may be straight chained or branched and may be in the Z or E form. Such groups include vinyl, propenyl, 1-butenyl, isobutenyl, 2-butenyl, 1-pentenyl, (Z)-2-pentenyl, (E)-2-pentyl, (Z)-4-methyl-2-pentenyl, (E)-4-methyl-2-pentenyl, allyl, pentadienyl, e.g., 1, 3 or 2,4-pentadienyl, and the like. It is preferred that the alkenyl group contains at most two carbon-carbon double bonds; and most preferably one carbon-carbon double bond.

The term alkynyl include alkynyls containing 2 to 6 carbon atoms. They may be straight chain as well as branched. It includes such groups as ethynyl, propynyl, 1-butynyl, 2-butynyl, 1-pentynyl, 2-pentynyl, 3-methyl-1-pentynyl, 3-pentynyl, 1-hexynyl, 2-hexynyl, 3-hexynyl, and the like.

The term aryl refers to an aromatic group containing only carbon ring atoms which contains up to 18 ring carbon atoms and up to a total of 25 carbon atoms and includes the polynuclear aromatic rings. These aryl groups may be monocyclic, bicyclic, tricyclic, or polycyclic, and contain fused rings. The group includes phenyl, naphthyl, anthracenyl, phenanthranyl, xylyl, tolyl and the like.

The aryl lower alkyl groups include, for example, benzyl, phenethyl, phenpropyl, phenisopropyl, phenbutyl, diphenylmethyl, 1,1-diphenylethyl, 1,2-diphenylethyl and the like.

The term halo include fluoro, chloro, bromo, iodo and the like.

The preferred values of R₂ and R₃ are independently hydrogen or alkyl containing 1-3 carbon atoms. It is most preferred that R₃ is hydrogen. It is most preferred that R₂ is hydrogen or alkyl containing 1-3 carbon atoms, especially methyl or ethyl. It is most preferred that R₂ is hydrogen or alkyl containing 1-3 carbon atoms or hydrogen and R₃ is hydrogen.

Alternatively, if R₂ and R₃ are taken together with the carbon atoms to which they are attached, it is most preferred that they form a phenyl ring. The phenyl ring is preferably unsubstituted or substituted with lower alkoxy, hydroxy, lower alkyl or halo.

It is preferred that R₄ is an electron withdrawing group, more specifically, halo, especially chloro, or is hydroxy or lower alkoxy. It is even more preferred that when R₁ is NHR₁₃, R₄ is substituted on the 7-position of the quinoline ring. It is most preferred that when R₁ is NHR₁₃, R₄ is halo.

However, when R₁₂ is NHR₁₃, it is preferred that R₄ is an electron donating group, such as hydroxy or alkoxy. More specifically, it is preferred that R₄ is methoxy or ethoxy when R₁₂ is NHR₁₃. It is even more preferred that R₄ is on the 6-position of the quinoline ring when R₁₂ is NHR₁₃.

It is preferred that one of R₅ and R₆ is hydrogen and the other is lower alkyl. It is even more preferred that R₅ is hydrogen and RA is lower alkyl, especially alkyl containing 1-3 carbon atoms and most preferably methyl.

The preferred value of R₇ is lower alkyl, especially alkyl containing 1-3 carbon atoms and most preferably methyl and ethyl.

Preferred values of R₈ is lower alkyl containing 1-3 carbon atoms, and most preferably methyl and ethyl. However, it is preferred that the alkyl group is unsubstituted or if substituted, is substituted on the omega (last) carbon in the alkyl substituent. The preferred substituent is lower alkoxy and especially hydroxy.

The preferred R₉ is lower alkoxy and especially hydroxy.

R₁₁ is preferably an electron withdrawing group, especially trifluoromethyl. It is preferably located on the 8-position of the quinoline ring.

R₁₄ is preferably an electron withdrawing group, and more preferably trifluoromethyl. It is preferably present on the 2-position of the quinoline ring.

It is preferred that R₁₅ is

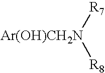

wherein R₇ and R₈ are independently alkyl containing 1-3 carbon atoms and Ar is phenyl.

In both R₁₃ and R₁₅, it is preferred that R₇ and R₈ contain the same number of carbon atoms, although one may be unsubstituted while the other is substituted. It is also preferred that R₇ and R₈ are the same.

The preferred value of n is 3 or 4 while the preferred value of n₁ is 1.

Preferred anti-malarials have the structure:

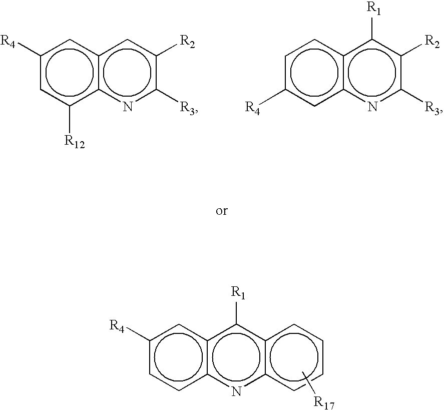

wherein R₁₂, R₄, R₂, R₃ and R₁ are as defined hereinabove and R₁₇ is hydrogen, halo, lower alkyl, lower alkoxy.

Preferred anti-malarials include the 8-aminoquinolines, 9-aminocridines and the 7-chloro-4-aminoquinolines. Examples include pamaquine, primaquine, pentaquine, isopentaquine, quinacrine salts, 7-chloro-4-aminoquinolines, such as the chloroquines, hydroxychloroquines, sontoquine, amodiaquine and the like.

Another class of preferred anti-malarial are cinchono alkaloids and 4-quinoline methanols, such as those having the formula:

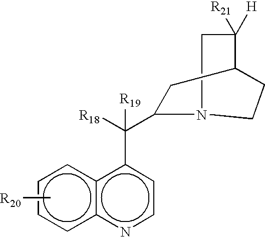

wherein one of R₁₈ and R₁₉ is hydroxy or loweralkylcarbonyloxy or hydrogen, and the other is H, and R₂₀ is hydrogen or loweralkoxy and R₂, is hydrogen or CH═CH₂.

Examples include rubane, quinine, quinidine, cinchoidine, epiquinine, epiquinidine, cinchonine, and the like.

Another preferred anti-malarial methanol is mefloquine or derivative thereof of the formula:

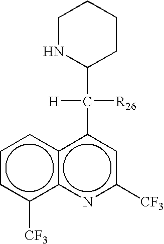

wherein R₂₆ is lower alkoxy,

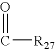

or hydroxy and R₂₇ is lower alkyl.

The most preferred anti-malarials include mefloquinine, and chloroquine and its congeners, such as hydroxychloroquine (HCQ), amodiaquine, pamaquine and pentaquine and pharmaceutically acceptable salts thereof.

The most preferred anti-malarial agent for the invention is hydroxychloroquine, shown below, or a pharmaceutically suitable salt thereof, such as hydroxychloroquine sulfate:

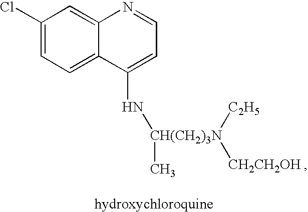

The anti-malarials are commercially available or are prepared by art recognized techniques known in the art.

For example, the 4-aminoquinolines can be prepared as follows:

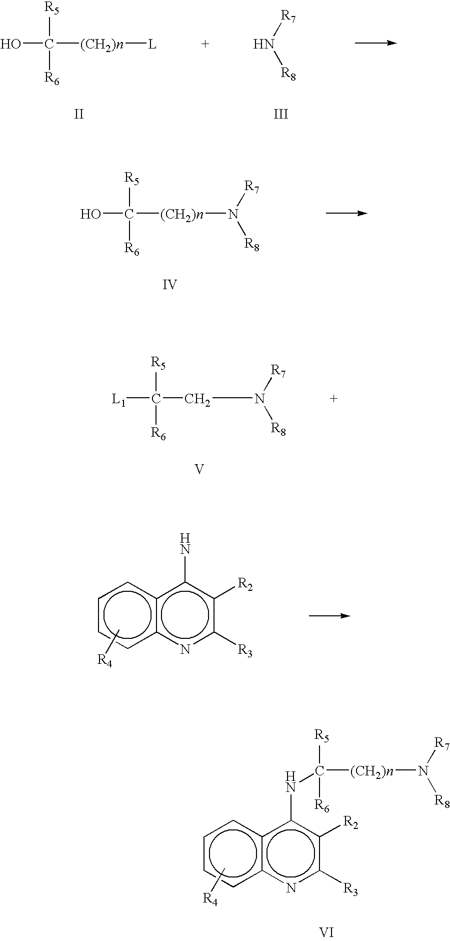

In the above scheme, R₁, R₂, R₃, R₄, R₅, R₆ R₇, R₈, and n are as defined hereinabove, and L and L₁ are good leaving groups, such as halides or sulfonates, e.g., mesylates or aryl sulfonates, e.g., tosylates, brosylates, and the like.

The compound of Formula II containing a leaving group, L, is reacted with the amine of Formula III under amine alkylation conditions. The alcohol group in the product of Formula IV (OH group) is converted to a leaving group by reactions known in the art. For example, sulfonic esters, such as tosylates, mesylates or brosylates are prepared by treatment of sulfonic halides of the formula R₂₃SO₂X₁, wherein X₁ is halide and R₂₃ is lower alkyl, such as methyl, aryl or substituted aryl, such as p-bromophenyl, p-tolyl with the alcohol of Compound IV. The reaction is usually effected in the presence of a weak base, such as pyridine. Alternatively, the alcohol can be converted to the corresponding halide by reaction of the alcohol of IV with HCl, HBr, thienyl chloride, PCl₃, PCl₅ or POCl₃. The product of V is then reacted under amine alkylation conditions with the quinoline amine to provide the 4-amino quinoline product.

The 9-aminoacridines and the 8-aminoquinoline are prepared similarly. More specifically, the product of V is reacted with

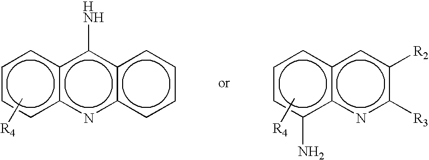

under amine alkylation reaction conditions.

The reactions described hereinabove are preferably conducted in solvents which are inert to the reactants and products and in which the reactants, are soluble, such as tetrahydrofuran, ethers, acetones, and the like. It is preferred that the solvents are volatile. The reactions are conducted at effective reaction conditions and are conducted at temperatures ranging from room temperature up to and including the reflux temperatures of the solvent.

An exemplary procedure for the preparation of compounds of Formula VII is as follows:

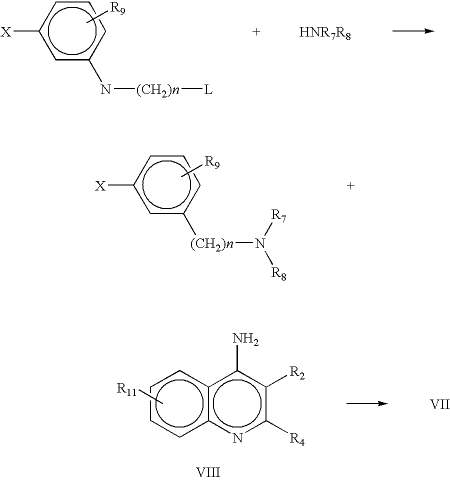

The first reaction is a simple amino alkylation reaction as described hereinabove. The product thereof is reacted with the amine of Formula III in the presence of a strong base such as amide to form the product of Formula VII.

Many of the compounds described hereinabove, especially the 4-quinoline methanols, can be converted to ethers by reacting the salt of the alcohols with an alkyl halide or arylalkyl halide or aryl halide to form the corresponding ether. Moreover, the esters can be formed from the hydroxy group by reacting the alcohol, such as the 4-quinoline methanol, with an alkanoic acid, arylalkonic acid or aryloic acid or acylating derivatives thereof in the presence of acid, for example, HCl, H₂SO₄ or p-toluene sulfonic acid under esterification conditions.

If any of the groups on R₁, R₂, R₃, R₄, R₅, R₆, R₇, R₈ are reactive with any of the reagents used or with any of the reactants or products, then they would be protected by protecting groups known in the art to avoid unwanted side reactions. This protecting groups normally used in synthetic organic chemistry are well known in the art. Examples are found in PROTECTIVE GROUPS IN ORGANIC SYNTHESIS, by T. W. Greene, John Wiley & Sons, Inc., NY 1981 (“Greene”), the contents of which are incorporated by reference.

Therapeutical Compositions of the Invention

A therapeutic composition within the present invention is formulated for controlled directed enteric delivery and includes at least one anti-malarial agent, as described above. As previously emphasized, the present invention contemplates topical administration of the anti-malarial compounds to the intra-luminal bowel wall where they may be absorbed with direct local therapeutic effect. “Directed enteric delivery” and “topical administration” are used in this description to denote direct delivery to the affected tissues or areas of diseased bowel. Controlled and “targeted delivery” when used together denotes the formulation of drug with excipient and/or carrier in such a way as to facilitate drug delivery to a specific organ of the gastrointestinal tract or e.g., colon, small bowel, and the like or portion thereof. “Sustained release” or synonym thereto connotes the release of the drug over a prolonged period of time. “Controlled delivery” denotes formulation of drug with carrier in such a way as to block absorption of drug in the proximal small bowel and facilitate drug delivery to inflamed areas of the more distal small bowel and/or colon. Carrier formulations which use controlled release technologies designed to delay drug release dependent on pH, transit time, or amount of hydration, or on the absence or presence of other physicochemical variables including biochemical markers of active inflammatory processes are included in this definition.

The anti-malarial compounds used in the present invention are administered in anti-inflammatory amounts. The anti-malarial compounds used in the present invention are administered in an amount which depends upon the condition of the subject, the type of inflammatory condition of which the subject suffers, the timing of the administration of the subject, the route of administration, the particular formulation and the like. However, unlike oral dosing, which takes three to six months for therapeutic effects, controlled directed enteric therapy will provide observable onset of action within two weeks. Significantly less amount of the active therapeutic moiety is needed to achieve these more rapidly achieved therapeutic benefits. It is preferred that the drug be administered at a total dose of about 2 to about 40 mg/day in one or more divided doses (0.05-10% conventional dosing).

While it is possible for the anti-malarial compound to be administered alone, it is preferable to present in a pharmaceutical formulation. The formulations used in the present invention comprise at least one anti-malarial compound according to the present invention together with one or more acceptable carriers thereof and optionally other therapeutic agents. Each carrier must be “acceptable” in the sense of being compatible with the other ingredients of the formulation and not injurious to the patient. The formulations may conveniently be presented in unit dosage form and may be prepared by any methods well known in the art of pharmacy. Such methods include the step of bringing into association the active ingredient with the carrier which constitutes one or more accessory ingredients. In general, the formulations are prepared by uniformly and intimately bringing into association the active ingredient with liquid carriers or finely divided solid carriers or both, and then if necessary shaping the product.

Formulations suitable for oral administration may be presented as discrete units such as capsules, cachets or tablets each containing a predetermined amount of the active ingredient; as a powder or granules. Oral formulations may further include other agents conventional in the art, such as sweeteners, flavoring agents and thickeners.

A tablet may be made by compression or moulding, optionally with one or more accessory ingredients. Compressed tablets may be prepared by compressing in a suitable machine the anti-malarials in a free-flowing form such as a powder or granules, optionally incorporating a binder (e.g., povidone, gelatin, hydroxypropylmethyl cellulose), lubricant, inert diluent, disintegrant (e.g. sodium starch glycollate, cross-linked povidone, cross-linked sodium carboxymethyl cellulose), surface-active or dispersing agent. Moulded tablets may be made by moulding in a suitable machine a mixture of the powdered compounds moistened with an inert liquid diluent.

The oral formulations are prepared so as to provide a targeted and controlled release of the anti-malarials in the esophagus, small intestine, large intestine, colon or rectum with minimal or no release in the stomach. Preferably, the anti-malarial is associated in a slow release formulation, such as e.g., tablet, so as to provide delayed or controlled release of the anti-malarials in the region having a pH relatively near the neutral range, with the additional property that it would transit into the more distal colon or small bowel. For example, the drug is formulated with a delayed drug release dependent on transit time, amount of hydration or the presence or absence of other physiochemical variables including biochemical markers of active inflammatory processes.

The pharmaceutical compositions of the present invention comprise one or more excipients and/or carriers known in the pharmaceutical arts which delay the release of the anti-malarial drug at the desired target in the gastrointestinal tract. The identity of the specific excipient or carrier is dependent upon several factors including the disease or condition of the patient being treated, the specific area in the gastrointestinal tract where the drug is targeted, to name just a few. The specific excipient or carrier to be used for the purpose of delaying the release at a specific targeted site is well within the knowledge of the skilled artisan.

In addition, the release of the anti-malarial compound may be immediate, i.e., the release may be delayed until the drug reaches the targeted site, but than the release is immediate. On the other hand, the present invention also contemplates sustained release formulation, wherein the pharmaceutical composition, besides comprising the anti-malarial compound, and carrier or excipient targeted for a specific site in the body, may also contain a sustained release carrier or excipient, e.g., sustained release polymer, to prolong the release thereof over a period of time. The pharmaceutical composition may comprise one or more sustained or controlled release excipients or carriers, such that a slow or sustained release of the anti-malarial compound is achieved. A wide variety of suitable excipients are known in the art. Such sustained/controlled release excipients, and systems are described for example, in U.S. Pat. Nos. 5,612,053, 5,554,387, 5,512,297,5,478,574 and 5,472,271, the contents of each of which is incorporated by reference. The anti-malarial compound of the present invention may be administered to a patient suffering from BD in the drug delivery device described in U.S. Pat. No. 4,904,474 to Theeves, the contents of which are incorporated by reference.

The anti-malarial compounds disclosed in the present application may be associated with a drug delivery system commercially marketed as OROS®, by ALZA corporation, for example, OROS®; Push-Pull™ System, or OROS® multi-layer, push Pull System, or OROS®, Push Stick System. Alternatively, the anti-malarial described herein may be associated with a sustained release formulation marketed as GEOMATRIX® which contains a combination of layers, each with different rates of swelling, gelling and erosion.

For instance, the anti-malarial compounds may be associated with a male piece and a female piece, with the pieces fitting together to enclose the anti-malarial therein, wherein the male piece is comprised of a material that gels in the intestinal juice, such as ethyl-acrylate-methyl methacrylate-trimethyl-ammonioethyl methacrylate chloride copolymer and a methacrylic acid-ethyl acrylate copolymer, while the female piece is made from a water insoluble polymer, as described in U.S. Pat. No. 6,303,144 to Omura, the contents of which are incorporated by reference.

Other drug delivery technologies include a coated bead system using MODAS: multiporous oral drug absorption system.

MODAS is a single unit, immediate release tablet formulation surrounded by a non-disintegrating, timed release coating. Within the gastro-intestinal tract this coating is transformed into a semi-permeable membrane through which drug diffuses in a rate-limiting manner. The diffusion process essentially dictates the rate of presentation of drug to the gastrointestinal fluids so that uptake into the body is controlled. Each MODAS tablet initially begins as a core containing active drug plus excipients. This is then coated with a solution of insoluble polymers and soluble excipients. Once the tablet is ingested the fluid of the gastrointestinal tract dissolves the soluble excipients in the outer coating leaving just the insoluble polymer. What results is a network of tiny, narrow channels connecting fluid from the GI tract to the inner drug core of water soluble drug. This fluid passes through these channels, into the core, dissolves the drug and a resultant solution of drug diffuses out in a controlled manner to the outside. This allows for controlled dissolution and absorption. The fact that the drug releasing pores in the tablet are distributed over the entire surface of the tablet facilitates uniform drug absorption and ensures that aggressive unidirectional drug delivery with its attendant hazards cannot occur.

MODAS represents a very flexible dosage form in that both the inner core and the outer semi-permeable membrane can be altered to suit the individual drug delivery requirements of a drug. In particular, the addition of excipients to the inner core such as buffers, etc., can help produce a micro-environment within the tablet that facilitates more predictable release rates and absorption.

The benefits of MODAS include:

(1) Ability to reduce the dosage frequency of highly water soluble drugs

(2) Smooth plasma profiles devoid of exaggerated peak to trough ratios

(3) Small size dosage forms due to minimal use of excipients.

Another drug delivery technology includes: PRODAS—Programmable Oral Drug Absorption System, based on the encapsulation of controlled release minitablets in the size range 1.5 to 4 mm in diameter. This consists of a hybrid of Multiparticulate and hydrophilic matrix tablet technologies and incorporates into one dosage form the benefits of both these drug delivery systems. The value of lies in the inherent flexibility of the formulation whereby combinations of mini-tablets, each with different release rates, are incorporated into a single dosage form.

These combinations may include immediate release, delayed release and/or controlled release minitablets. For each individual compound therefore, the technology enables the construction of a customized release profile based on the use of different populations of mini-tablets each with different release rates.

As well as allowing for controlled absorption over a specified period, PRODAS also enables targeted delivery of drug to specified sites of absorption throughout the gastrointestinal tract. Combination products are also possible by using mini-tablets formulated with different active ingredients.

Another drug delivery technology includes SODAS —Spheroidal Oral Drug Absorption System, another multi-particulate drug delivery technology platform on which the company was initially founded.

Based on the production of controlled release beads, it is characterized by its inherent flexibility, enabling the production of dosage forms with customized release rates that respond directly to individual drug candidate needs.

The controlled release beads produced by the SODAS technology range from 1 to 2 mm in diameter. Each begins as a non-pareil core on to which a solution of active ingredient is applied. A series of subsequent coatings with timing solutions (containing both soluble and insoluble polymers) and other excipients combine to produce the outer rate controlling membrane that ultimately controls release of drug from the beads. Once produced, the controlled release beads can be packaged into a capsule or compressed into a tablet to produce the final dosage form.

Drug release from SODAS beads is by a diffusion process. Within the GI tract the soluble polymers dissolve leaving pores within the outer membrane. Fluid then enters the core of the beads and dissolves the drug. The resultant solution then diffuses out in a controlled, pre-determined manner allowing for prolongation of the in-vivo dissolution and absorption phases. As each candidate drug presents itself with different physicochemical properties the composition of the semi-permeable membrane will differ for each individual SODAS formulation.

In addition, the immediate environment of the drug within the seed core can be manipulated through use of excipients to ensure optimal stability and solubility.

The unique nature of the SODAS technology gives rise to a number of attributes that directly benefit individual drugs:

(1) Controlled absorption with resultant reduction in peak to trough ratios

(2) Targeted release of the drug to specific areas within the gastrointestinal tract

(3) Absorption irrespective of the feeding state

(4) Minimal potential for dose dumping

Another drug delivery technology is based on an agglomerated hydrophilic matrix. The matrix consists of two pharmaceutically accepted polysaccharides, locust bean gum and xanthan gum. Interactions between these components in an aqueous environment form a tight gel with slowly eroding core. This system controls the rate of water ingress into the matrix and the subsequent diffusion and release of the drug from the dosage form.

A further example is Microtrol™, a proven family of multiparticulate delivery technolgies which improve solubility and deliver a variety of modified release profiles. Microtrol is based on the use of beadlets that can be filled into capsules or compressed into tablets. The beadlets can be coated (with an array of controlled release polymers) or uncoated. Different combinations of badlets can be used to achieve customized release profiles. These include: extended delivery Mictrol XR; pulsed delivery Microtrol PR; and delayed delivery.

The anti-malarial compounds e.g., HCQ, may also be administered in a rectum applicable preparation known in the art such as in a suppository, enema and the like in a conventional form, such as a suspension, foam or liquid and the like. These rectum applicable preparations can be produced by conventional means, incorporating the anti-malarial compounds therein in effective amounts. These rectum applicable preparations can be used to treat inflammatory diseases involving the anus, proctosigmoid colon and distal colon (transverse and descending colon).

The anti-malarial compound is administered in the rectum applicable preparation so that an effective amount is administered to the patient. Preferably, the amount administered rectally to the patient is about 1 to about 10% of the anti-malarial compound that is administered conventionally orally. It is preferred that sufficient amount of anti-malarial compound is present in the rectum applicable preparation so that about 0.01 mole to about 0.1 mole of anti-malarial compound is ultimately administered to the patient in each rectal preparation.

As used herein, “pharmaceutically acceptable carrier” includes any and all solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredient, its use in the therapeutic compositions is contemplated. More than one anti-malarial compound can also be incorporated into the pharmaceutical compositions.

It is especially advantageous to formulate compositions in dosage unit form for ease of administration and uniformity of dosage. Dosage unit form as used herein refers to physically discrete units suited as unitary dosages for the mammalian subjects to be treated; each unit containing a predetermined quantity of anti-malarial compound calculated to produce the desire therapeutic effect in association with the required pharmaceutical carrier.

The above preferred embodiments are given to illustrate the scope and spirit of the present invention. The embodiments described herein will make apparent to those skilled in the art other embodiments. These other embodiments are within the contemplation of the present invention. Therefore, the present invention should be limited only by the appended claims.

What is claimed is:

1. A method for treating inflammatory bowel disease in a patient comprising administering to said patient a sustained release pharmaceutical composition comprising a pharmaceutically effective amount of an anti-malarial compound in association with a pharmaceutically acceptable excipient which delays and targets the release of said anti-malarial compound in the gastrointestinal tract of the patient.

2. The method according to claim 1 wherein the inflammatory bowel disease is Crohn's disease.

3. The method according to claim 1 wherein the inflammatory bowel disease is ulcerative colitis.

4. The method according to claim 1 wherein the inflammatory bowel disease is indeterminate colitis.

5. The method according to claim 1 wherein the inflammatory bowel disease is infectious colitis.

6. The method according to claim 1 wherein the anti-malarial compound is aminoquinoline or hydroxyquinoline.

7. The method according to claim 6 wherein said aminoquinoline has the formula: 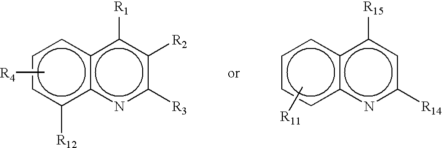
or pharmaceutically acceptable salts thereof, wherein
R₂ and R₃ are independently hydrogen, or lower alkyl or R₂ and R₃ taken together with the carbon atoms to which they are attached form an aryl ring, which aryl ring is unsubstituted or substituted with an electron withdrawing group or an electron donating group,
one of R₁ and R₁₂ is NHR₁₃ while the other is hydrogen; 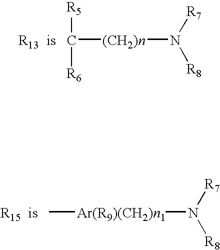
R₄, R₁₀, R₁₁ and R₁₄ are independently hydrogen or an electron donating group or electron withdrawing group;
R₅ and R₆, are independently hydrogen or lower alkyl which may be unsubstituted or substituted with an electron withdrawing or electron donating group;
R₇ and R₈ are independently hydrogen or lower alkyl, which may be unsubstituted or substituted with an electron withdrawing or electron donating group;
Ar is aryl having 6-18 ring carbon atoms which may be unsubstituted or substituted with an electron donating or electron withdrawing group;
R₉ is hydrogen or hydroxy or lower alkoxy or 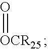
R₂₅ is lower alkyl or hydrogen; and
n and n, are independently 1-6.

8. The method according to claim 7 wherein the aminoquinoline is of the formula:

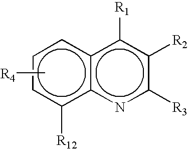

9. The method according to claim 8 wherein R₁ is NHR₁₃ and R₁₂ is hydrogen.

10. The method according to claim 9 wherein R₅ is hydrogen and R₆ is lower alkyl.

11. The method according to claim 9 wherein R₅ is hydrogen and R₆ is methyl.

12. The method according to claim 9 wherein n is 3.

13. The method according to claim 9 wherein R₃ is hydrogen.

14. The method according to claim 9 wherein R₄ is substituted in the 7-position of the quinoline ring.

15. The method according to claim 11 wherein R₄ is 7-halo.

16. The method according to claim 15 wherein halo is chloro.

17. The method according to claim 9 wherein R₇ is ethyl and R₈ is ethyl or 2-hydroxy ethyl.

18. The method according to claim 8 wherein R₁₂ is NHR₁₃ and R₁ is hydrogen.

19. The method according to claim 18 wherein R₅ is hydrogen and R₆ is lower alkyl.

20. The method according to claim 19 wherein R₅ is hydrogen and R₆ is methyl.

21. The method according to claim 18 wherein n is 3.

22. The method according to claim 19 wherein
R₇ is hydrogen, methyl or ethyl and R₈ is hydrogen, methyl, ethyl, propyl or isopropyl.

23. The method according to claim 18 wherein R₄ is substituted on the 6-position of the quinoline ring.

24. The method according to claim 23 wherein R₄ is 6-lower alkoxy.

25. The method according to claim 24 wherein R₄ is 6-methoxy.

26. The method according to claim 7 wherein the amino quinoline has the formula:

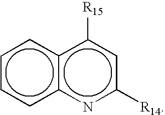

27. The method according to claim 26 wherein Ar is phenyl.

28. The method according to claim 26 wherein R₉ is hydroxy.

29. The method according to claim 26 wherein R₁₅ is

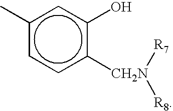

30. The method according to claim 26 wherein R₇ and R₈ are independently lower alkyl.

31. The method according to claim 30 wherein R₇ and R₈ are both ethyl

32. The method according to claim 1 wherein the anti-malarial compound has the formula: 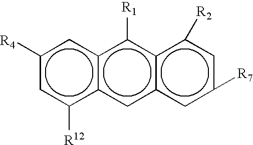
wherein
R₂ is hydrogen or lower alkyl;
one of R₁ and R₁₂ is NHR₁₃ while the other is hydrogen; 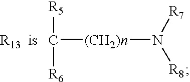
R₄ is hydrogen or an electron donating group or electron withdrawing group;
R₅ and R₆, are independently hydrogen or lower alkyl which may be unsubstituted or substituted with an electron withdrawing or electron donating group;
R₇ and R₈ are independently hydrogen or lower alkyl, which may be unsubstituted or substituted with an electron withdrawing or electron donating group; and
n is independently 1-6.

33. The method according to claim 1 wherein the anti-malarial agent is pomaquine, primaquine, pentaquinine, isopentaquine, quinacrine salt, chloroquine, hydroxychloroquine, sontoquine, amodiaquine, mefloquine, or mepacrine or pharmaceutically acceptable salts thereof.

34. The method according to claim 1 wherein the anti-malarial compound is hydroxychloroquine, chloroquine, mepacrine, mefloquinine, or pharmaceutically acceptable salts thereof.

35. The method according to claim 1 wherein the anti-malarial compound is hydroxychloroquine or a pharmaceutically acceptable salt thereof.

36. A pharmaceutical composition comprising a pharmaceutically effective amount of an anti-malarial compound in association with a pharmaceutically acceptable excipient which delays and targets the release of said anti-malarial compound in the gastrointestinal tract.

37. The pharmaceutical composition according to claim 36 wherein the anti-malarial compound is aminoquinoline or hydroxyquinoline.

38. The pharmaceutical composition according to claim 37 wherein said aminoquinoline has the formula: 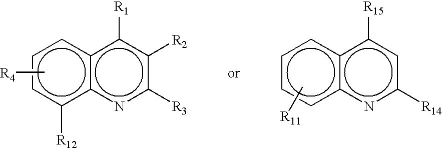
or pharmaceutically acceptable salts thereof, wherein
R₂ and R₃ are independently hydrogen, or lower alkyl or R₂ and R₃ taken together with the carbon atoms to which they are attached form an aryl ring, which aryl ring is unsubstituted or substituted with an electron withdrawing group or an electron donating group,
one of R₁ and R₁₂ is NHR₁₃ while the other is hydrogen; 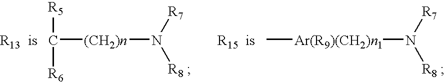
R₄, R₁₀, R₁₁ and R₁₄ are independently hydrogen or an electron donating group or electron withdrawing group;
R₅ and R₆, are independently hydrogen or lower alkyl which may be unsubstituted or substituted with an electron withdrawing or electron donating group;
R₇ and R₈ are independently hydrogen or lower alkyl, which may be unsubstituted or substituted with an electron withdrawing or electron donating group;
Ar is aryl having 6-18 ring carbon atoms which may be unsubstituted or substituted with an electron donating or electron withdrawing group;
R₉ is hydrogen or hydroxy or lower alkoxy or 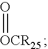
R₂₅ is lower alkyl or hydrogen; and
n and n₁, are independently 1-6.

39. The pharmaceutical composition according to claim 38 wherein the aminoquinoline is of the formula:

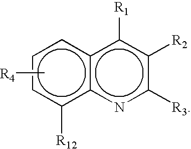

40. The method according to claim 39 wherein R₁ is NHR₁₃ and R₁₂ is hydrogen.

41. The method according to claim 40 wherein R₅ is hydrogen and R₆ is lower alkyl.

42. The method according to claim 40 wherein R₅ is hydrogen and R₆ is methyl.

43. The method according to claim 40 wherein n is 3.

44. The method according to claim 40 wherein R₃ is hydrogen.

45. The method according to claim 40 wherein R₄ is substituted in the 7-position of the quinoline ring.

46. The method according to claim 40 wherein RA is 7-halo.

47. The pharmaceutical composition according to claim 46 wherein
halo is chloro.

48. The pharmaceutical composition according to claim 40 wherein R₇ is ethyl and R₈ is ethyl or 2-hydroxy ethyl.

49. The pharmaceutical composition according to claim 39 wherein R₁₂ is NHR₁₃ and R₁ is hydrogen.

50. The pharmaceutical composition according to claim 49 wherein R₅ is hydrogen and R₆ is lower alkyl.

51. The pharmaceutical composition according to claim 50 wherein R₅ is hydrogen and R₆ is methyl.

52. The pharmaceutical composition according to claim 49 wherein n is 3.

53. The pharmaceutical composition according to claim 50 wherein R₇ is hydrogen, methyl or ethyl and R₈ is hydrogen, methyl, ethyl, propyl or isopropyl.

54. The pharmaceutical composition according to claim 49 wherein R₄ is substituted on the 6-position of the quinoline ring.

55. The pharmaceutical composition according to claim 54 wherein R₄ is 6-lower alkoxy.

56. The pharmaceutical composition according to claim 55 wherein R₄ is 6-methoxy.

57. The pharmaceutical composition according to claim 38 wherein the amino quinoline has the formula:

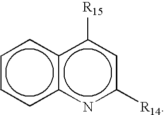

58. The pharmaceutical composition according to claim 57 wherein Ar is phenyl.

59. The pharmaceutical composition according to claim 57 wherein R₉ is hydroxy.

60. The pharmaceutical composition according to claim 57 wherein R₁₅ is

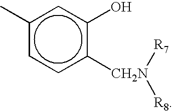

61. The pharmaceutical composition according to claim 57 wherein R₇ and R₈ are independently lower alkyl.

62. The pharmaceutical composition according to claim 61 wherein R₇ and R₈ are both ethyl

63. The pharmaceutical composition according to claim 36 wherein the anti-malarial compound has the formula: 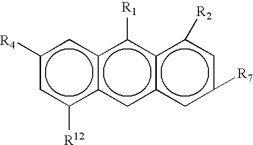
wherein
R₂ is hydrogen or lower alkyl;
one of R₁ and R₁₂ is NHR₁₃ while the other is hydrogen; 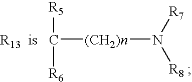
R₄ is hydrogen or an electron donating group or electron withdrawing group;
R₅ and R₆, are independently hydrogen or lower alkyl which may be unsubstituted or substituted with an electron withdrawing or electron donating group;
R₇ and R₈ are independently hydrogen or lower alkyl, which may be unsubstituted or substituted with an electron withdrawing or electron donating group; and
n is independently 1-6.

64. The pharmaceutical composition according to claim 36 wherein the anti-malarial agent is pomaquine, primaquine, pentaquinine, isopentaquine, quinacrine salt, chloroquine, hydroxychloroquine, sontoquine, amodiaquine, mefloquine, or mepacrine or pharmaceutically acceptable salts thereof.

65. The pharmaceutical composition according to claim 36 wherein the anti-malarial compound is hydroxychloroquine, chloroquine, mepacrine, mefloquinine, or pharmaceutically acceptable salts thereof.

66. The pharmaceutical composition according to claim 36 wherein the anti-malarial compound is hydroxychloroquine or a pharmaceutically acceptable salt thereof.

67. The method according to claim 1 wherein the inflammatory bowel disease is eosinophilic gastroenteritis.

68. A method of preventing or treating an inflammatory bowel disease characterized by eosinophilic tissue infiltration caused by or resulting from ulcerative colitis, eosinophilic gastroenteritis, Crohn's disease, eosinophilic esophagitis, ileitis, proctosigmoiditis, or proctitis; which comprises
administering a pharmaceutically effective amount of an anti-malarial compound to a patient in need thereof to suppress said eosinophilic tissue infiltration.

69. The method according to claim 1 wherein the anti-malarial compound is administered rectally as a rectum applicable preparation.

70. The method according to claim 69 wherein the anti-malarial compound is HCQ.

71. The pharmaceutical composition according to claim 37 which is a rectal applicable preparation.

72. The pharmaceutical composition according to claim 71 wherein the anti-malarial compound is HCQ.

73. A method for treating eosinophilic esophagitis in a patient suffering from same which comprises administering to said patient an anti-malarial compound in an amount effective to treat said eosinophilic esophagitis in association with a carrier which promotes release thereof in the esophagus.

74. The method according to claim 73 wherein the anti-malarial compound is HCQ.
